(19) United States
(12) Patent Application Publication
(10) Pub. No.: US 2023/0256136 A1
(43) Pub. Date: Aug. 17, 2023

(54) ANTIMICROBIAL SILK NANOPARTICLES AND METHODS FOR MAKING AND USING THE SAME

(71) Applicant: University of Georgia Research Foundation, Inc., Athens, GA (US)

(72) Inventors: Hitesh HANDA, ATHENS, GA (US); Sama GHALEI, Athens, GA (US)

(21) Appl. No.: 18/018,275

(22) PCT Filed: Jul. 28, 2021
(86) PCT No.: PCT/US2021/043365

§ 371 Date: Jan. 27, 2023

Related U.S. Application Data
(60) Provisional application No. 63/057,335, filed on Jul. 28, 2020.

(51) Int. Cl. A61L 26/00

(52) U.S. Cl. CPC A61L 26/0066; A61L 26/008; A61L 26/0085; A61L 26/0047; A61L 2300/114; A61L 2300/406; A61L 2300/624; A61L 2400/12; B82Y 5/00

(57) ABSTRACT

Described herein are biocompatible materials that include a nitric oxide (NO) donor embedded in silk fibroin nanoparticles. In one aspect, the nitric oxide donor is present in the hydrophobic core of the silk fibroin nanoparticles such that the nitric oxide donor is encapsulated. The biocompatible materials described herein serve as a biocompatible and inexpensive nitric oxide delivery platform that provide sustained release of nitric oxide. The biocompatible materials are non-toxic and can be used in biomedical applications such as wound healing, where a combination of therapeutic and antibacterial properties of silk and nitric oxide are desired. Additionally, described herein are methods of making the biocompatible materials.

### CROSS-REFERENCE TO RELATED APPLICATIONS

This application claims priority to and the benefit of U.S. provisional application Ser. No. 63/057,335 filed Jul. 28, 2020, the contents of which are incorporated by reference in its entirety.

### STATEMENT REGARDING FEDERALLY SPONSORED RESEARCH OR DEVELOPMENT

This invention was made with Government support under contract HL134899 awarded by the National Institutes of Health. The Government has certain rights in the invention.

### BACKGROUND

Nitric oxide (NO) is a diatomic free radical gas molecule in humans and other mammals, which exhibits various functions in physiological processes including inhibition of platelet adhesion and activation, prevention of infection, regulation of angiogenesis and wound repair [1, 2]. NO is endogenously produced by nitric oxide synthase (NOS) enzymes at nM and μM concentrations [1]. The multifaceted roles of NO have inspired researchers to develop NO-releasing/generating materials that can provide an exogenous supply of NO for different biomedical applications [3]. For instance, NO releasing coatings have been successfully developed to prevent blood clots in blood-contacting devices and implants to inhibit platelet activation and further thrombosis formation [4]. Moreover, NO has emerged as a potent antibacterial agent against a broad spectrum of bacteria. NO is known to kill the bacteria by reacting with oxygen or reactive oxygen intermediates and formation of peroxynitrite (—OONO), which is a cytotoxic compound against invading pathogens [5, 6]. In addition to antibacterial effects, NO has also been recognized to play an essential role in wound-healing by regulating angiogenesis, cell proliferation, and tissue remodeling [7]. Despite the beneficial functions of nitric oxide, the gaseous radical state and short half-life of NO have restricted its direct delivery [8].

### SUMMARY

Described herein are biocompatible materials that include a nitric oxide (NO) donor embedded in silk fibroin nanoparticles. In one aspect, the nitric oxide donor is present in the hydrophobic core of the silk fibroin nanoparticles such that the nitric oxide donor is encapsulated. The biocompatible materials described herein serve as a biocompatible and inexpensive nitric oxide delivery platform that provide sustained release of nitric oxide. The biocompatible materials are non-toxic and can be used in biomedical applications such as wound healing, where a combination of therapeutic and antibacterial properties of silk and nitric oxide are desired. Additionally, described herein are methods of making the biocompatible materials.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D are SEM (FIGS. 1A and 1C) and TEM (FIGS. 1B and 1D) images of (FIGS. 1A and 1B) silk fibroin nanoparticles (SF NPs) and (FIGS. 1C and 1D) SNAP-silk fibroin nanoparticles (SNAP-SF NPs) in accordance with embodiments of the present disclosure.

FIG. 2 provides FTIR spectra of SF NPs and SNAP-SF NPs in accordance with embodiments of the present disclosure.

FIGS. 3A-3B provide deconvoluted FT-IR spectra of the amide I band of (3A) SF NPs and (3B) SNAP-SF NPs in accordance with embodiments of the present disclosure. The crystallinity content for these samples are 16% and 23% respectively. B=β-sheets.

FIG. 4 illustrates example storage stability of SNAP-SF NPs at different conditions over 4 weeks.

FIG. 5 graphs 24 h viability of 3T3 mouse fibroblast cells (as percentage relative to control) using WST dye-based CCK-8 assay. Error bar represents standard deviation (n=6). P-values ≤01 and ≤5 are shown with * and **, respectively.

FIGS. 6A-7B plot antibacterial activity of SF NPs and SNAP-SF NPs in accordance with embodiments of the present disclosure against (7A) MRSA and (7B) E. coli after 24 h.

FIG. 7 shows live/dead imaging of 3T3 mouse fibroblast cells at different concentrations of NPs. Live cells are stained green by calcein AM and dead cells are stained red by EthD-III. The same control was used for both SF NPs and SNAP-SF NPs. Scale bar=400 μm.

FIG. 8 shows the cumulative and real-time NO release from SNAP-SF NPs soaking in PBS buffer with or without collagenase type II, at 37° C. in the dark. * and ** indicate p-values ≤05 and ≤01, respectively.

FIGS. 9A-10B show SEM (A) and TEM (B) images of SNAP-SF NPs loaded PLA nanofibers. TEM image shows the homogenous spreading of the nanoparticles in a PLA nanofiber.

FIG. 10 shows the real-time NO release profile of the SNAP-SF NPs loaded PLA fibers.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions and Abbreviations

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an inert excipient” includes, but are not limited to, mixtures or combinations of two or more such inert excipients, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “silk fibroin” is an insoluble protein present in silk produced by numerous insects, such as the larvae of Bombyx mori, and other moth genera such as Antheraea, Cricula, Samia and Gonometa. In one aspect, the silk fibroin used to the produce the biocompatible materials described herein is derived from Bombyx mori cocoons.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The term “antimicrobial effective amount” as used herein refers to that amount of the compound being administered which will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

“Alginate” as used herein refers to the salts of alginic acid (usually sodium alginate), but it can also refer to alginic acid or derivatives of alginic acid. Alginate, also called algin, is a natural polymer present in the cell walls of brown algae.

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to the entire host or organism and not just a part excised (e.g., a liver or other organ) from the living host.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “substituted” refers to any one or more hydrogens on the designated atom that can be replaced with a selection from the indicated group, provided that the designated atom's normal valence is not exceeded.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof

The term “substituted,” as in “substituted alkyl”, means that the substituted group may contain in place of one or more hydrogens a group such as alkyl, hydroxy, amino, halo, trifluoromethyl, cyano, —NH(alkyl), –N(alkyl)₂, alkoxy, alkylthio, or carboxy, and thus embraces the terms haloalkyl, alkoxy, fluorobenzyl, and the sulfur and phosphorous containing substitutions referred to below.

The terms “amine” and “amino” are art-recognized and refer to both unsubstituted and substituted amines, e.g., a moiety that can be represented by the general formula:

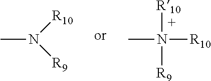

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “alkyl amino group” is an alkyl group as defined herein substituted with one or more amino groups.

Abbreviations: NO, nitric oxide; SF NPs, silk fibroin nanoparticles; SNAP, S-Nitroso-N-acetylpenicillamine; SNAP-SF NPs, SNAP-loaded Silk fibroin nanoparticles.

### Discussion

In accordance with the purpose(s) of the present disclosure, as embodied and broadly described herein, embodiments of the present disclosure, in some aspects, relate to biocompatible materials that include a nitric oxide (NO) donor embedded in silk fibroin nanoparticles.

The biocompatible materials described herein include silk fibroin and a NO donor. In some embodiments the biocompatible material includes silk fibroin nanoparticles substantially encapsulating the NO donor in the hydrophobic core of the nanoparticle. Advantageously, the nanoparticles have both antimicrobial properties and can be non-cytotoxic. Because the silk fibroin is a natural polymer capable of biocompatibility, tunable biodegradation, and water-based processing, it can be used in various applications such as wound healing and bone tissue engineering. The encapsulation of the NO donor provides for silk fibroin particles with improved antimicrobial and antithrombotic properties.

The silk fibroin nanoparticles described herein include a shell (or exterior area) comprised of hydrophilic molecular segments and a core (or interior area) comprising hydrophobic molecular segments that encapsulate the NO donor by hydrophobic interactions, as the NO donor is water-insoluble and slightly hydrophobic. This specific conformation is obtained through the method of making the nanoparticles, described in further detail below. In one aspect, the silk fibroin nanoparticles are a matrix composed of hydrophobic and hydrophilic polymeric chains, wherein the hydrophilic chains are located near the outer side of the particles and the hydrophobic segments form the core/interior of the nanoparticles. During fabrication of the nanoparticles, most of the slightly hydrophobic NO donor becomes entrapped in the hydrophobic core. The term “encapsulation” is used to describe the tendency for the NO donor to accumulate in the hydrophobic center of the silk fibroin nanoparticles; however some additional NO donor may be present on the surface of the nanoparticles. Thus, a majority of the NO donor is embedded in the hydrophobic core of the nanoparticles, where greater than 50%, greater than 55%, greater than 60%, greater than 65%, greater than 70%, greater than 75%, greater than 80%, greater than 85%, greater than 90%, or greater than 95% of the NO donor in the biocompatible materials described herein is present in the core of the silk fibroin nanoparticles.

In one aspect, the nitric oxide donor is an S-nitroso thiol of formula O═N—S—R, where R can be an alkyl or aryl moiety. Reference to alkyl and aryl moieties includes substituted and unsubstituted alkyl and aryl moieties, respectively. In an aspect, the alkyl, substituted alkyl, aryl, or substituted aryl moiety can comprise from about 5 to about 20 carbons. In another aspect, the nitric oxide donor can be an amino acid moiety with a thiol group. In another embodiment, the nitric oxide donor can be an S-nitroso thiol. In one aspect, the nitric oxide donor is S-nitroso-N-acetyl-penicillamine, S-nitroso-N-acetyl cysteine, S-nitroso-N-acetyl cysteamine, S-nitrosoglutathione, methyl S-nitrosothioglycolate, or any combination thereof. In another aspect, the S-nitroso thiol is S-nitroso-N-acetylpenicillamine (SNAP), derivatives or salts thereof, S-Nitroso-glutathione, derivatives or salts thereof. In another aspect, the nitric oxide donor includes an organic nitrate, a metal-NO complex, or an N-nitrosamine.

Described herein are methods for making the biocompatible materials. In one aspect, the method involves (a) mixing a nitric oxide donor composition comprising a nitric oxide donor and a binary solvent with a silk fibroin to form a first composition comprising the biocompatible material and (b) isolating the biocompatible material from the first composition.

The nitric oxide donor composition is produced by mixing the nitric oxide in a binary solvent. In one aspect, the binary solvent comprises an alcohol (e.g., methanol, ethanol, isopropanol, propanol) and water. In one aspect, the binary solvent is about 20:80 to about 40:60 by volume alcohol/water, or about 20:80, 25:75, 30:70, 35:65, or 40:60. In other aspects, the binary solvent can include other organic solvents besides alcohols including, but not limited to, acetone, and DMSO.

The silk fibroin can be added to the nitric oxide donor composition as a solid or as a composition. In one aspect, the silk fibroin is formulated in water then subsequently mixed with the nitric oxide donor composition. In one aspect, the ratio of NO donor to silk fibroin is about 2:1 to about 1:4 by mass, or about 2:1, 1.5:1, 1:1, 1:1.5, 1:2, 1:2.5; 1:3, 1:3.5, or 1:4, where any value can be a lower and upper endpoint of a range (e.g., 1.5:1 to 1:1.5). After mixing the silk fibroin with the nitric oxide donor composition, the resulting composition can be incubated at from about 0° C. to about −40° C. to produce the biocompatible material.

In one aspect, the biocompatible material is an emulsion after the silk fibroin is mixed with the nitric oxide donor composition. In one aspect, the emulsion is centrifuged to produce a sediment, and the sediment is subsequently freeze-dried to produce a powder comprising silk fibroin nanoparticles embedded with the nitric oxide donor. The nanoparticles can be stored for extended periods of time until ready for use. Nonlimiting procedures for preparing the biocompatible materials described herein are provided in the Examples.

The encapsulation efficiency as used herein describes the total amount (weight) of nitric oxide donor per initial amount of nitric oxide donor in the nitric oxide donor composition. In one aspect, the encapsulation efficiency using the methods described herein can be about 15% to 20% or about 15%, 15.5%, 16%, 16.5%, 17%, 17.5%, 18%, 18.5%, 19%,19.5%, or 20%, where any value can be a lower and upper endpoint of a range (e.g., 15.5% to 19%).

The loading capacity as used herein describes the amount (weight) of nitric oxide donor released per total weight of the nitric oxide donor nanoparticles. In one aspect, the nitric oxide donor is from about 5% by weight to about 15% by weight of the biocompatible material, or about 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15%, where any value can be a lower and upper endpoint of a range (e.g., 5.5% to 12%).

The particle size of the biocompatible materials described herein can vary depending upon the application of the nanoparticles. In one aspect, the biocompatible material has a diameter of about 300 nm to about 500 nm, or about 300 nm, 325 nm, 350 nm, 400 nm, 425 nm, 450 nm, 475 nm, or 500 nm, where any value can be a lower and upper endpoint of a range (e.g., 350 nm to 450 nm).

In one aspect, the biocompatible materials described herein possess an overall negative charge. In one aspect, the biocompatible material described herein has a negative charge of about −25 mV to about −30 mV, or about −25 mV, −25.5 mV, −26 mV, −26.5 mV, −27 mV, −27.5 mV, −28 mV, −28.5 mV, −29 mV, −29.5 mV, or −30 mV, where any value can be a lower and upper endpoint of a range (e.g., −26 mV to −28 mV).

The biocompatible materials described herein possess good release patterns that make them useful in biomedical applications. In one aspect, the release half-life of the nitric oxide donor from the biocompatible material is about 2 hours to about 8 hours, or about 2 hours, 2.5 hours, 3 hours, 3.5 hours, 4 hours, 4.5 hours, 5 hours, 5.5 hours, 6 hours, 6.5 hours, 7 hours, 7.5 hours, or 8 hours, where any value can be a lower and upper endpoint of a range (e.g., 2.5 hours to 6 hours). In another aspect, the amount of nitric oxide release from the biocompatible material is about 0.010 nmol/mg to about 0.20 nmol/mg in about 24 hours, or about 0.010 nmol/mg, 0.020 nmol/mg, 0.030 nmol/mg, 0.040 nmol/mg, 0.050 nmol/mg, 0.060 nmol/mg, 0.070 nmol/mg, 0.080 nmol/mg, 0.090 nmol/mg, 0.10 nmol/mg, 0.11 nmol/mg, 0.12 nmol/mg, 0.13 nmol/mg, 0.14 nmol/mg, 0.15 nmol/mg, 0.16 nmol/mg, 0.17 nmol/mg, 0.18 nmol/mg, 0.19 nmol/mg, or 0.20 nmol/mg, where any value can be a lower and upper endpoint of a range (e.g., 0.12 nmol/mg to 0.18 nmol/mg). Advantageously, this release rate is more controlled than other materials that have a NO release half-life of less than two hours. Not wishing to be bound by theory, the improved release rate of the nitric oxide donor from the biocompatible materials described herein is due at least in part to the hydrophobic interior area of the silk fibroin nanoparticles limiting the diffusion of the nitric oxide donor.

The biocompatible materials described herein can include one or more additional drugs (e.g. pain relievers like NSAIDs, antibiotics, anticancer drugs). In one aspect, the drug can be added to the nitric oxide donor composition then subsequently mixed with the silk fibroin to incorporate the drug into the nanoparticles. In another aspect, a composition composed of the silk fibroin and drug can be added to the nitric oxide donor composition to incorporate the drug into the nanoparticles.

The biocompatible materials described herein have numerous biomedical applications. In one aspect, the biomedical materials can be used in would healing. For example, the biocompatible materials described herein can be incorporated into hydrogels, which can subsequently used in wound dressings. In one aspect, the biocompatible materials described herein can be mixed with one or more polymers and water to produce hydrogels. For example, the biocompatible material can be mixed with alginate solution and crosslinked to make an alginate hydrogel containing the biocompatible material. The hydrogel can include polymers such as, for example, alginate, gelatin, polyethylene glycol, polyvinyl alcohol, a poloxamer, or any combination thereof. The hydrogel can be an amorphous gel or can be incorporated into an article such as a wound dressing having an adhesive layer and/or a barrier material.

In another aspect, the biocompatible materials described herein can be included in a matrix material or scaffold. In one aspect, the matrix material can be a hydrogel, sponge, polymeric film, nanofibers, etc. Polymeric solutions including solvents that do not dissolve the biocompatible materials described herein can be used to form a matrix. In one aspect, a solution of the biocompatible material and a polymer such as, for example, polylactic acid can be electrospun or cast to form polymeric nanofibers or films containing the biocompatible materials described herein.

In other aspect, the biocompatible materials described herein can also be included in other articles where controlled release of an antimicrobial is desired (e.g., sponges).

### Aspects

Aspect 1. A biocompatible material comprising a nitric oxide donor embedded in silk fibroin nanoparticles.

Aspect 2. The biocompatible material according to Aspect 1, wherein the nitric oxide donor is a S-nitrosothiol compound.

Aspect 3. The biocompatible material according to Aspect 2, wherein the S-nitrosothiol compound is S-nitroso-N-acetyl-penicillamine, S-nitroso-N-acetyl cysteine, S-nitroso-N-acetyl cysteamine, S-nitrosoglutathione, methyl S-nitrosothioglycolate, or any combination thereof.

Aspect 4. The biocompatible material according to any one of Aspects 1-3, wherein the silk fibroin nanoparticles comprise a hydrophobic core, wherein a majority of the nitric oxide donor is present in the hydrophobic core of the silk fibroin nanoparticles.

Aspect 5. The biocompatible material according to any one of Aspects 1-4, wherein the silk fibroin nanoparticles have a diameter of about 300 nm to about 500 nm.

Aspect 6. The biocompatible material according to any one of Aspects 1-5, wherein the ratio of nitric oxide donor to silk fibroin nanoparticles is about 2:1 to about 1:4 by mass.

Aspect 7. The biocompatible material according to any one of Aspects 1-6, wherein a release half-life of the nitric oxide donor from the biocompatible material is about 2 hours to about 8 hours.

Aspect 8. The biocompatible material according to any one of Aspects 1-7, wherein the amount of nitric oxide donor release from the biocompatible material is about 0.010 nmol/mg to about 0.20 nmol/mg in about 24 hours.

Aspect 9. The biocompatible material according to any one of Aspects 1-8, wherein the nitric oxide donor is from about 5% by weight to about 15% by weight of the biocompatible material.

Aspect 10. The biocompatible material according to any one of Aspects 1-9, wherein the biocompatible material has a negative charge of about −25 mV to about −30 mV.

Aspect 11. The biocompatible material according to any one of Aspects 1-10, wherein the biocompatible material further comprises a drug.

Aspect 12. A biocompatible material produced by the method comprising:
mixing a nitric oxide donor composition comprising a nitric oxide donor and a binary solvent with a silk fibroin to form a first composition comprising the biocompatible material; and
isolating the biocompatible material from the first composition.

Aspect 13. The biocompatible material according to Aspect 12, further comprising centrifuging the first composition to obtain a sediment; and
freeze-drying the sediment to obtain a powder comprising silk fibroin nanoparticles embedded with the nitric oxide donor.

Aspect 14. The biocompatible material according to Aspect 12 or 13, wherein the S-nitrosothiol compound is S-nitroso-N-acetyl-penicillamine, S-nitroso-N-acetyl cysteine, S-nitroso-N-acetyl cysteamine, S-nitrosoglutathione, methyl S-nitrosothioglycolate, or any combination thereof.

Aspect 15. The biocompatible material according to Aspect 12 or 13, wherein the nitric oxide donor is S-nitroso-N-acetylpenicillamine.

Aspect 16. The biocompatible material according to any one of Aspects 12-15, wherein the binary solvent comprises an alcohol and water.

Aspect 17. The biocompatible material according to any one of Aspects 12-15, wherein the binary solvent comprises ethanol and water.

Aspect 18. The biocompatible material according to Aspect 17, wherein a ratio of ethanol to water in the binary solvent is about 20:80 to about 40:60 by volume.

19. An article comprising the biocompatible material according to any one of Aspects 1 to 18.

Aspect 20. The article of Aspect 19, wherein the article is a wound dressing comprising a hydrogel.

Aspect 21. The article of Aspect 19, wherein the article is a sponge.

Aspect 22. A hydrogel comprising the biocompatible material according to any one of Aspects 1 to 18.

Aspect 23. The hydrogel of Aspect 22, wherein the hydrogel comprises alginate, gelatin, polyethylene glycol, polyvinyl alcohol, a poloxamer, or any combination thereof.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

Silk cocoons from Bombyx mori (B. mori) were purchased from paradise fibers (USA). Absolute ethanol was purchased from VWR (USA), and Spectra/Por™ 3 RC dialysis membrane tubing (3.5 KDa, MWCO) was purchased from fisher scientific (USA). N-Acetyl-D-penicillamine (NAP), lithium bromide, calcium chloride, potassium chloride, sodium chloride, potassium phosphate monobasic, sodium phosphate dibasic, tetrahydrofuran (THF), ethylenediaminetetraacetic acid (EDTA), sulfuric acid, and N,N dimethylacetamide were purchased from Sigma Aldrich (St. Louis, Mo. 63103). LB broth was obtained from Fisher Bioreagents (Fair Lawn, N.J.). LB Agar was purchased from Difco Laboratories Inc. (Detroit, Mich.). Phosphate-buffered saline (PBS), pH 7.4, containing 138 mM NaCl, 2.7 mM KCl, and 10 mM sodium phosphate, was used for all in vitro experiments. Dulbecco's modified Eagle's medium (DMEM) and trypsin-EDTA were purchased from Corning (Manassas, Va. 20109). The Cell Counting Kit-8 (CCK-8) was obtained from Sigma Aldrich (St. Louis, Mo. 63103). The antibiotic Penicillin-Streptomycin (Pen-Strep) and fetal bovine serum (FBS) were purchased from Gibco-Life Technologies (Grand Island, N.Y. 14072). The Cu-NPs (99%, 40-60 nm) were obtained from SkySpring Nanomaterials, Inc. (Houston, Tex.). The bacterial strains methicillin-resistant Staphylococcus aureus (MRSA) and E. coli (ATCC 25922), and mouse 3T3 cells (ATCC 1658) were originally obtained from American Type Culture Collection (ATCC).

### Synthesis of Silk Fibroin and SNAP

Aqueous silk fibroin solution was isolated from Bombyx mori cocoons following standard extraction procedure [27]. Briefly, B. mori cocoons were degummed by boiling in 20 mM sodium carbonate solution for 30 min and air dried. The dried silk fibroin was dissolved in 9.3 M lithium bromide at 60° C. for 4 h and dialyzed in a 3.5 KDa Cut-off membrane against distilled water for 48 h. The dialyzed solution was then filtered and stored at 4° C. for further use.

SNAP was synthesized using a modified version of a previously reported method [28]. Briefly, an equimolar amount of sodium nitrite and NAP were added to a 1:1 mixture of methanol and water containing 1 M H₂SO₄ and 1 M HCl. The mixture was stirred for 30 min in the dark and then cooled in an ice bath for 5 h to precipitate the SNAP crystals. The collected crystals were rinsed, dried under vacuum in the dark, and were stored at −20° C. for use in all experiments.

### Synthesis of Silk and SNAP Loaded Silk NPs

SNAP-SF NPs were prepared using the previously described anti-solvent/self-assembling method [29]. SNAP was first dissolved in 30 mL binary solvent mixture of ethanol/water at 30:70 v/v. This mixture was then added dropwise to 10 mL aqueous SF solution (2% wt/v) under gentle stirring. The mixed solution was incubated in a refrigerator at −20° C. for exactly 3 h and was defrosted at room temperature to turn into a milky emulsion. The NPs emulsion was centrifuged at 4700 rpm for 10 min, the supernatant was discarded, and the pellet was re-suspended in deionized water (DIW). The NPs sediment was washed 3 times to remove any unloaded SNAP and freeze-dried at −80° C. for 48 h to obtain SNAP-SF NPs powder. The particles were stored at −20° C. until use. Fabrication of pure SF NPs was carried out using the same procedure as SNAP-SF NPs. However, the freezing time was increased to 24 h to allow enough time for the formation of pristine silk NPs. It has been reported that SF NPs only form within a narrow range of ethanol-SF ratio (10:90-50:50). However, in this study at lower or higher ratios than 30:70, SF NPs either did not form or made a gel/aggregated.

### Size and Zeta Potential Analysis

The size and surface charge of the NPs in DI water were analyzed using Dynamic Light Scattering (DLS) (ZetaSizer, Malvern). Appropriate dilutions were carried out before analysis to meet the requirement of the equipment. Experiments were conducted at 25° C. at a fixed angle of 60° .

### Morphology Observation

Morphology of the nanoparticles was observed using a field emission scanning electron microscope (SEM, FEI Teneo, FEI Co.) at an accelerating voltage of 5.00 kV. The NPs emulsion was sonicated for 5 min and diluted with deionized water before FE-SEM observation. TEM samples were prepared by dropping 2 μL of the diluted nanoparticles dispersion on carbon coated electron microscopy grids. The specimens were air dried in dust free condition before examination under JEOL JEM-2100 transmission electron microscope. (voltage applied: 200 kV). Size analysis was performed with ImageJ software.

### Fourier-Transform Infrared Spectroscopy (FTIR)

The FTIR spectra of the SF NPs and SNAP-SF NPS (in the form of KBr pellets) were analyzed using a Nicolet 6700 spectrometer (Thermo Electron Corporation, Madison, Wis.). For each measurement, the spectra were obtained from 128 scans with a resolution of 4 cm⁻¹ over the wavenumber range of 4000-400 cm⁻¹. The crystallinity (β-sheet content) of SF in SF NPs and SNAP-SF NPs was measured by deconvolution of amide I band using OriginPro 8 software according to previous studies [30]. Different contributions were fitted using Gaussian-shaped peaks, using an equal, fixed width for all the considered peaks. The crystallinity content was calculated from the ratio between the areas of the b-sheets peaks and the total area of the amide I peak.

### Measurement of NO Loading and Release

NO release from SNAP-SF NPs was measured via chemiluminescence using a Sievers Nitric Oxide Analyzer (NOA) model 280i (Boulder, Colo.). Samples were weighed and incubated in 0.01 M PBS containing EDTA at 37° C. in an amber reaction vessel to protect from light. Nitric oxide released from the suspension of particles was continuously swept from the vessel by an N₂ purge gas at a flow rate of 200 mL min⁻¹ into the NOA system. The NO levels in ppb unit measured in each time point were converted to NO release rate in mol min⁻¹ mg⁻¹ unit using the NOA instrument constant, determined by quantitative reduction of a known amount of nitrite. Between the final and initial NOA measurements, physiological conditions were maintained by storing the samples in 0.01 M PBS with EDTA at 37° C.

To measure the total NO loading, alternating injections of 0.25 M copper (II) chloride and ascorbic acid were added to the NOA reaction vessel. These injections triggered the release of all NO present in the sample within a time frame measurable by NOA. The total amount of NO released was calculated by the integration of NO release rate curves. As each SNAP molecule contains one NO molecule, the total moles of NO released can be considered equal to the moles of SNAP present in the nanoparticles. Therefore, the number of NO molecules can be converted to the weight of their associated donor molecules based on SNAP molecular weight. Accordingly, encapsulation efficiency (EE) and loading capacity (LC) values of NO in SNAP-SF NPs was determined based on the initial predetermined SNAP loading in the nanoparticles using Eq. (1) and Eq. (2), respectively.

$$EE ( \% ) = \frac{Total amount of NO released ( mg SNAP )}{Initial amount of NO ( mg SNAP )} \times 100 \qquad ( 1 )$$

$$LC ( \% ) = \frac{Total amount of NO released ( mg SNAP )}{Total Weight of SNAP - SF NPs ( mg )} \times 100 \qquad ( 2 )$$

### Cell Culture and Viability Studies

3T3 mouse fibroblast cells (ATCC 1658) were cultured in a 75 cm² T-flask containing complete DMEM medium with 10% fetal bovine serum (FBS) and 1% penicillin-streptomycin to prevent contamination. The T-flask with cells was incubated at 37° C., in a 5% CO₂ humidified environment to allow for monolayer formation. The culture medium was changed every two days, and cells were checked daily for growth and contamination. After the confluency reached above 80%, cells were trypsinized (0.18% trypsin and 5 mM EDTA) and detached from the T-flask. Finally, the cells were counted under a hemocytometer using Trypan blue (dye exclusion method).

The effect of nanoparticles on cell viability was determined by the CCK-8 cell counting kit according to the manufacture's protocol (Sigma Aldrich). The CCK-8 test is nondestructive in nature and more sensitive than other tetrazolium salts such as MTT, XTT, WST-1, and MTS. The number of living cells is directly proportional to the amount of formazan dye (orange color) generated by the interaction of the highly water-soluble tetrazolium salt, WST-8, with dehydrogenases in the cells and is detected at the absorbance maxima of 450 nm. First, cells were seeded in 96-well plates at a density of 3000 cells per well and allowed to attach in a humidified incubator with 5% CO₂ for 24 h. The media were then replaced with fresh media containing SF NPs or SNAP-SF NPs at various concentrations (0.25, 0.5, 1, 2, 5, and 10 mg/mL) and incubated for another 24 hours. Cells maintained in DMEM without nanoparticles were used as a control group. To avoid absorbance interference by SF and SNAP-SF NPs, the media containing nanoparticles was removed and 100 μL fresh media plus 10 μL of CCK-8 solution was added into each well and incubated for an additional 1 h at 37° C. The absorbance was detected at 450 nm. The data were expressed as mean ±standard deviation (SD) of five replicates (n=5), and the cell viability was calculated using the following equation:

$$Cell viability ( \% ) = \frac{Absorbance ( treated cells )}{Absorbance ( control cells )} \times 100 \qquad ( 3 )$$

### In Vitro Bacterial Viability

To assess the antibacterial efficacy of the SF NPs and SNAP-SF NPs, E. coli (ATCC 25922) and methicillin-resistant Staphylococcus aureus MRSA (ATCC BAA 041) was used. For both, LB broth was used for inoculation and incubated for 12 h at 37° C. under shaking conditions (150 rpm). The bacterial cultures were grown to a mid-log phase. The resulting bacteria solution was centrifuged at 2500 rpm for 7 min. The supernatant was discarded, and the bacteria re-suspended in PBS and re-centrifuged to get rid of dead cells and other cell debris. The resulting bacterial pellet was again re-suspended in PBS and adjusted to ˜10⁸ CFU/mL. In a 96-well plate, different concentrations of each sample were exposed to the final bacterial solution. Post-exposure, the 96-well plate was incubated for 24 h at 37° C. at 150 rpm (n=3). To evaluate the log reduction and reduction efficiency of bacteria, bacterial solution from each well was collected and serially diluted (10⁻¹-10⁻⁵) and plated on LB agar and incubated for 24 h. CFU counted provided the number of bacteria per mL of solution. Percentage reduction in bacterial viability was calculated from the following equation where,

$$C = \frac{CFU}{mL} :$$

$$Reduction of bacterial viability ( \% ) = \frac{{C}_{control} - {C}_{sample}}{{C}_{control}} \times 100 \qquad ( 4 )$$

### Storage Stability Evaluation

SNAP-SF NPs were placed in vials under the following conditions: room temperature with ambient light, room temperature in the dark, 37° C. in the dark, and in the freezer (−20° C.) in the dark. At various time points over a month, NO content in the nanoparticles was measured to determine the % SNAP remaining in them. This was compared to the initial amount of SNAP loaded into nanoparticles calculated by total NO release measurement immediately after fabrication [13].

### Statistical Analysis

All data are reported as a mean±standard deviation collected from three data points unless otherwise noted. A one-way analysis of variance was used to compare any significant difference. P-values of <0.5 were considered statistically significant.

### Results and Discussion

### Synthesis and Characterization of SNAP-SF NPs

SF NPs were produced using a bottom-up approach called anti-solvent precipitation that builds nanoparticles by assembling silk molecular chains together [31]. The addition of ethanol, which is a poor solvent for aqueous silk fibroin, causes a conformational transition from the random coil and/or helical to β-sheet structures in SF molecular chains to some degree. The formed b-sheet nanocrystals then grow into nanoparticles under shearing force induced during the freezing step. The SF NPs produced by this method feature a hydrophobic interior composed of well-ordered crystalline β-sheet structures. Whereas the shell of the NPs consists of mainly hydrophilic segments [29]. Therefore, the hydrophobic core of SF NPs provides the right environment to encapsulate the water-insoluble and slightly hydrophobic SNAP molecules by hydrophobic interactions. This method has been previously used to load hydrophobic drugs such as Paclitaxel in the core of SF NPs for cancer treatment applications [32]. Advantageously, ethanol acts both as the solvent of SNAP and the additive for NPs formation, which makes the process simpler and cost-effective.

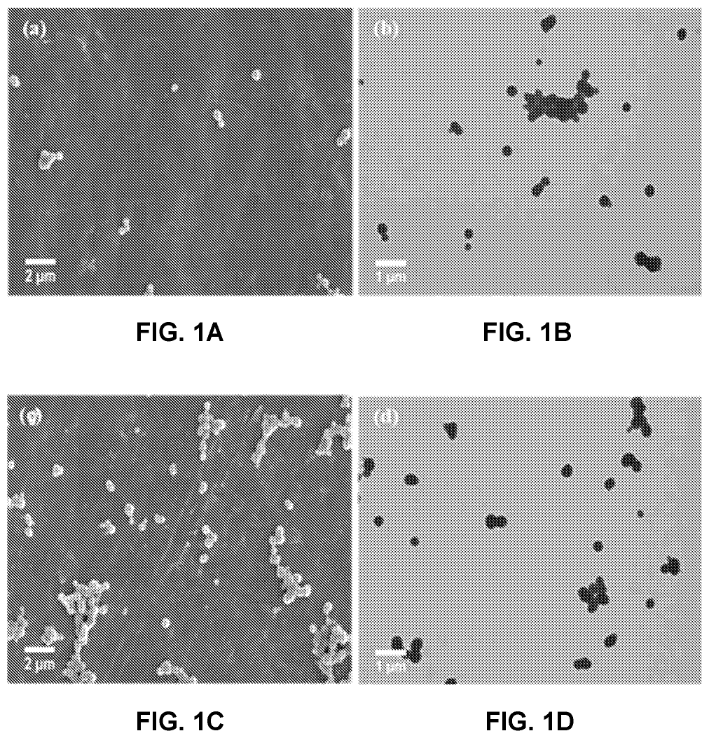

According to TEM, SF NPs produced by this method had an average diameter of 345.1±23.3 nm and a spherical morphology which was observed by SEM (FIG. 1). Previous studies have also reported a similar size range for SF NPs produced using this method [29]. SNAP-SF NPs showed the same morphology and were slightly larger than SF NPs with an average diameter of 382.5±20.1 nm, which is probably due to encapsulation of SNAP in the NPs. There was no evidence of excess crystallized SNAP on the surface of SNAP-SF NPs. The average size of NPs measured from TEM images was consistent with the results obtained from DLS studies (Table 1). The result of zeta potential analysis demonstrated that SF NPs and SNAP-SF NPs have an overall negative charge of ˜−26 and ˜−28 mV respectively at pH 7-8. The increase in NPs size with SNAP loading may have influenced the surface charge of the SF NPs. An increase in surface negativity has also been reported by Wang et al. for ibuprofen incorporation into SF nanoparticles [33]. Moreover, the high negativity of the NPs surface suggests that the suspension of NPs would be quite stable in DI water. Correspondingly, it was observed that freeze-dried NPs powder could be easily re-dispersed in aqueous media. Encapsulation efficiency (EE) and loading capacity (LC) values of NPs with different SNAP to SF ratios were measured using NOA, and the results are summarized in Table 2. EE and LC increased slightly by increasing ratio from 1:4 to 1:1, however, it decreased with further increasing the ratio. That is probably because, after a certain ratio of SNAP:SF, the polymer matrix gets saturated and cannot incorporate more drugs into it. Overall, 1:1 ratio of SNAP:SF had the highest EE and LC values, and therefore, it was chosen for further studies.

TABLE 1
Zeta-potential and particle sizes
of SF NPs and SNAP loaded SF NPs

| Sample | Zeta potential | Size (from DLS) |
|---|---|---|
| SF NPs | −26.41 ± 0.87 mV | 338.43 ± 7.10 nm |
| SNAP-SF NPs | −28.76 ± 0.73 mV** | 379.36 ± 15.92* nm |

TABLE 2
Loading capacity and encapsulation efficiency
in SNAP-SF NPs with different SNAP-to-SF ratios

| SNAP:SF | Encapsulation | Loading |
|---|---|---|
| Ratio | efficiency (%) | capacity (%) |
| 2:1 | 10.80 ± 1.8* | 3.60 ± 0.23 |
| 1:1 | 18.31 ± 1.32 | 9.15 ± 0.65** |
| 1:2 | 16.6 ± 1.14 | 5.35 ± 0.33 |
| 1:4 | 13.87 ± 1.01 | 2.77 ± 0.16 |

### FTIR and Deconvolution of Amide I Band

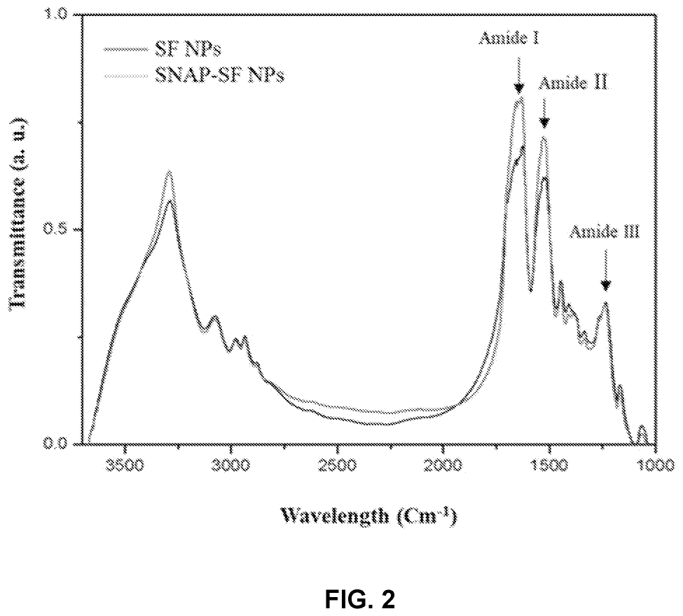

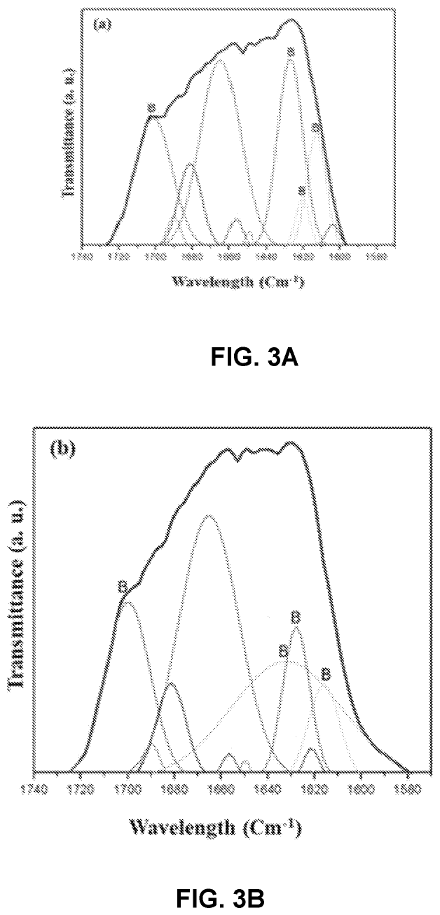

FTIR spectra of pristine SF NPs and SNAP-SF NPs are shown in FIG. 2. The amide I, II, and III characterization peaks of silk fibroin protein are the dominant peaks in both spectra and cover the SNAP characterization peaks. It can be seen that the absorption at the amide I band increases with the addition of SNAP. This might be attributed to the increase of the β-sheets conformation in SNAP-SF NPs [32, 34]. To characterize the modifications induced by SNAP addition, the amide I peak was analyzed by peak deconvolution and reported in FIG. 3. The crystallinity degree was calculated for each sample accordingly. As expected, SF NPs showed an average β-sheets content of 16%, while SNAP-SF NPs presented an average crystallinity degree of 23%. This means that SNAP addition is favorable for the conformation transition of SF from random coil/helix to β-sheet, probably because of the hydrophobic interactions between SNAP and SF macromolecules. In addition, the more β-sheet formation induced by SNAP may be beneficial to enhance its encapsulation in the SF NPs and tailor the release kinetics.

### NO Release Kinetics and Storage Stability

NO release from RSNOs can be triggered by thermal decomposition, catalysis (using metals ions such as Cu), or by exposure to light [35, 36]. In this study, NO release from SNAP-SF NPs was measured using PBS containing EDTA in an amber reaction vessel to protect from metal ions and light-induced NO release, respectively. Therefore, the main mechanism of NO release in this study is the decomposition of the SNAP entrapped in the SF NPs by means of moisture/heat via homolytic cleavage of the S—NO bond resulting in disulfide species (RSSR) formation (Eq. 5) [37].

2RSNO→RSSR+2NO (5)

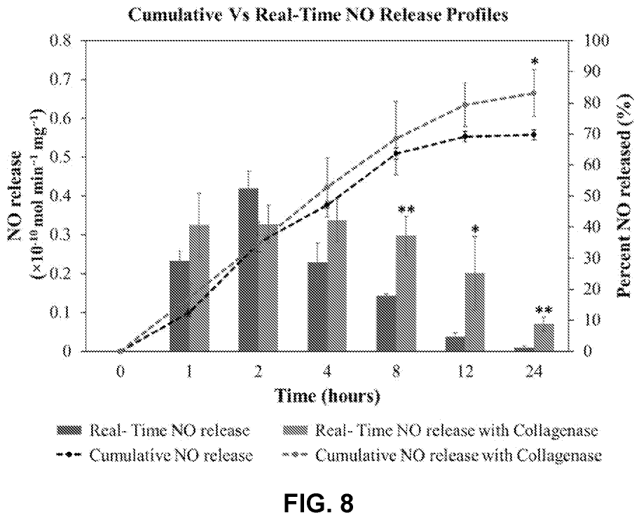

The NO release rate from SNAP-SF NPs at different time points is shown in FIG. 8 over a 24 hours period. The highest release rate equal to 0.042±0.043 nM was observed at 2 h, and after that, the measured NO release rates began to decrease with time until it reached ˜0.001 nM at 24 h. The control of SF NPs over NO release is shown by the estimated cumulative NO release curve (FIG. 8), which were obtained by integrating the real-time NO release rates with the trapezoidal rule. The NO release half-life was 4 h, which is a notable improvement compared to previously reported biopolymeric nanoparticle-based NO delivery systems that reportedly deliver more than half of their NO payload in less than 2 h [21, 22]. This might be attributed to the hydrophobic core of SF NPs that limits the diffusion of SNAP into the buffer due to minimal water uptake. Therefore, SNAP molecules entrapped inside the core will release at a slower rate. SNAP-SF NPs released up to 0.131±0.029 nmol/mg NO over a 24 h period before the concentration increase became undetectable.

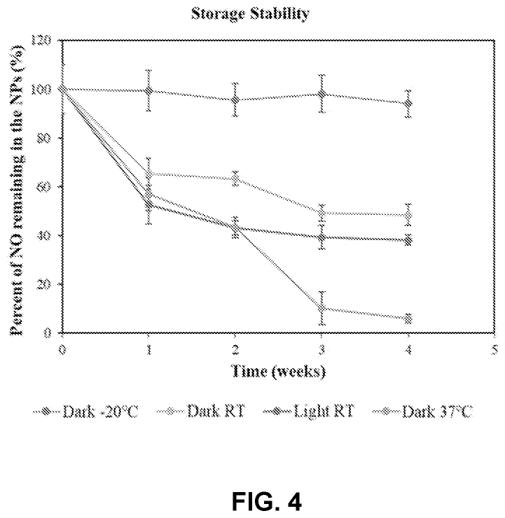

The storage stability of the SNAP-SF NPs was tested under different conditions (FIG. 4). SNAP-SF NPs showed excellent stability in −20° C. at dark conditions maintaining almost all their NO content after a month. However, SNAP-SF NPs kept at room temperature either at dark or light conditions, lost more than 30% of their NO after a week, and more than 50% after a month. Lowest stability was observed for SNAP-SF NPs kept at 37° C., which indicates the higher susceptibility of SNAP to release NO at elevated temperatures compared to light. Overall, the optimum storage condition was found to be at −20° C. in the dark, where 94% of SNAP was remaining after a month.

### Enzyme Mediated Nitric Oxide (NO) Release Profile:

SF is a protein based natural polymer and undergoes biodegradation by proteolytic enzymes, yielding non-toxic amino acids. Therefore, in addition to moisture/heat assisted NO release, the enzymatic biodegradation of SF NPs might also contribute to the final NO release kinetics. To study the effects of SF degradation process on NO release kinetics of SNAP-SF NPs, NO release from these NPs was measured in PBS containing collagenase type II at 37° C. The presence of collagenase in the media did not have any significant effect on the NO release from SNAP-SF NPs in the first 4 hours (FIG. 8). However, at 8 h, 12 h, and 24 h, SNAP-SF NPs in PBS containing collagenase released NO ata rate of 0.29±0.04×10⁻¹⁰ mol min⁻¹ mg⁻¹, 0.20±0.09×10⁻¹⁰ mol min⁻¹ mg⁻¹, and 0.07±0.01×10⁻¹⁰ mol min⁻¹ mg⁻¹, respectively, which was significantly higher than the real-time NO release rates from SNAP-SF NPs in PBS without collagenase at the same corresponding time points. This indicates that the SF proteolytic degradation process most likely starts after 8 h of incubation in collagenase solution, giving rise to higher NO release rates from SNAP-SF NPs. In addition, according to the cumulative NO release profiles (FIG. 8), the cumulative percentage of NO released from SNAP-SF NPs in media containing collagenase (83.09±7.50%) was significantly higher than that of SNAP-SF NPs in media without collagenase (69.86±1.57%).

### Cell Cytotoxicity Assay

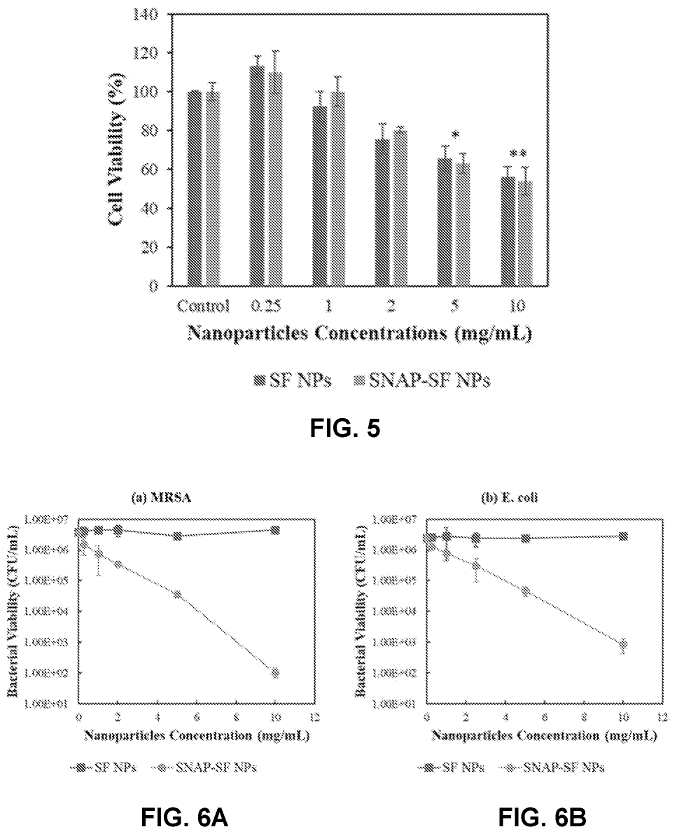

NO exhibits dose-dependent toxicity towards cells. It has been suggested that high concentrations of NO in the micro- to millimolar range is frequently associated with cell apoptosis and cytotoxic effects caused by nitrosative stress. Whereas, low local NO concentrations, in the pico- to nanomolar range, exerts beneficial effects on cellular proliferation and protection [38]. The cytocompatibility of SF and SNAP-SF NPs was evaluated in vitro in direct contact with L929 cells (FIG. 5). No apparent cytotoxicity was observed below 5 mg/mL concentrations for both SF NPs and SNAP-SF NPs. At 5 mg/mL, NPs showed weak cytotoxicity resulting in 65.8% and 63.14% cell viability when treated with SF NPs and SNAP-SF NPs, respectively. The cell viability values further reduced to 55.9% and 53.9% at 10 mg/mL concentration of SF NPs and SNAP-SF NPs, respectively. The viabilities of fibroblasts treated with SNAP-SF NPs were not significantly different from those treated with SF NPs at all concentrations. Therefore, it can be inferred that cytotoxicity of the NPs at higher concentrations (5 and 10 mg/mL) is not caused by SNAP, but by SF NPs itself. Since SF is known to be a biocompatible polymer, the cytotoxicity of SF NPs might be caused by the high number of NPs.

### Live/Dead Staining

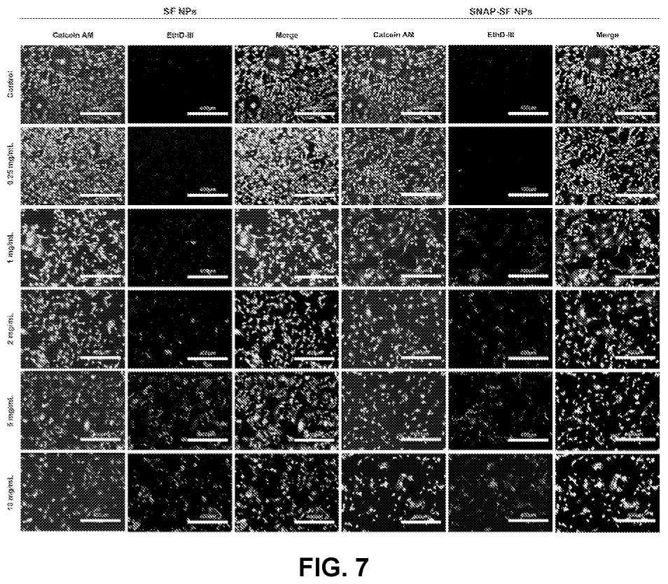

Viability of 3T3 mouse fibroblast cells treated with different concentrations of SF NPs and SNAP-SF NPs was assessed qualitatively by EthD-III/calcein AM staining. As shown in FIG. 7, cells treated with lower NPs concentrations (0.25-2 mg/mL) showed minimal decrease in viability when compared with untreated controls. However, exposure to higher concentrations of NPs (5 mg/mL and 10 mg/mL), resulted in a greater decrease in the number of live cells similar to the results obtained from the quantitative in vitro viability assay.

### In Vitro Bacterial Viability

Unlike antibiotics, NO is known to be a potent antimicrobial agent that does not develop resistance. Because it exerts antibacterial effects through multiple non-specific mechanisms. These mechanisms include chemical alteration of bacterial DNA, disturbing protein synthesis, and damaging cell membrane amino acids [39, 40]. In this study, the antibacterial activities of SF NPs and SNAP-SF NPs against MRSA and Escherichia coli (E. coli) were assessed by the CFU counting method (FIG. 5A and B). SNAP-SF NPs showed antibacterial activity against both bacteria in a concentration-dependent manner. There was a significant reduction of up to >2 logs (99.2% killing efficiency) and >4 logs (99.99% of killing efficiency) by 5 mg/mL and 10 mg/mL SNAP-SF NPs, respectively. It was also observed that the E. coli viability reduced by ˜2 logs (98.7% of killing efficiency) and ˜4 logs (99.96% of killing efficiency) using 5 mg/mL and 10 mg/mL SNAP-SF NPs, respectively. In contrast, SF NPs did not affect the viability of MRSA and E. coli regardless of the NPs concentration, suggesting that antibacterial activity of the SNAP-SF NPs can be accredited merely to the NO content.

NO can kill the bacteria by either directly modifying membrane-bound proteins, metabolic enzymes, and DNA, or by interacting with superoxides and producing reactive nitrogen species, which further alter functions of proteins that are critical for cellular processes [40, 41]. However, some of the bacterial species such as E. coli, have adapted themselves to nitrosative stress by regulating different genes and producing certain enzymes that help them neutralize the disrupting effects of NO to some extent [42]. Therefore, it was expected that the viability of E. coli would decrease less than MRSA when treated with the same amount of NO releasing SF NPs. Nevertheless, the sustained release of NO from SNAP-SF NPs likely overcame bacterial resistance mechanisms resulting in the high killing bactericidal effect.

### Preparation of Nanofibers

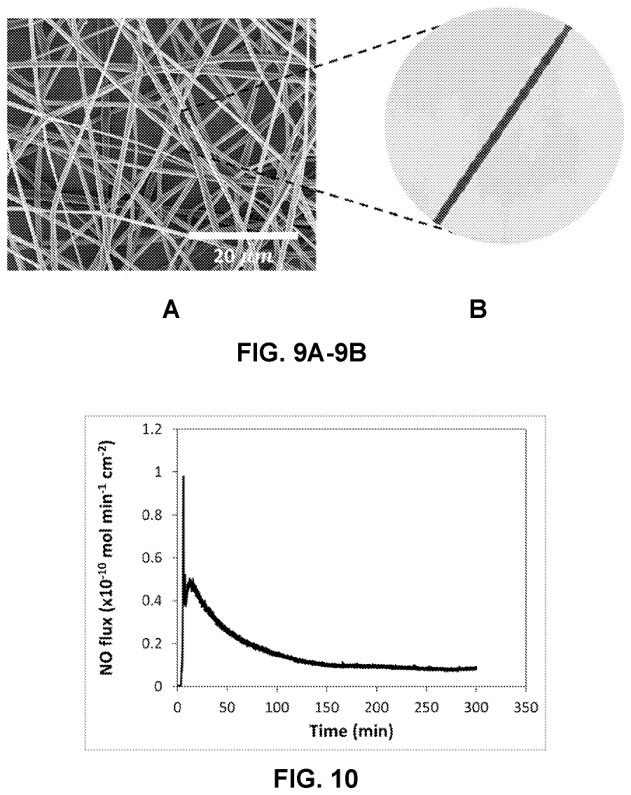

SNAP-SF NPs were incorporated into polylactic acid (PLA) polymeric nanofibers through electrospinning method. The scanning electron microscopy (SEM) and transmission electron microscopy (TEM) images showed successful incorporation of SNAP-SF nanoparticles in the PLA fibers (FIG. 9A-10B). The SNAP-SF NPs loaded PLA nanofibers had a sustained release of NO at a rate of ˜0.1 (×10⁻¹⁰ mol min⁻¹ cm⁻²) that was measured over 300 min (FIG. 10).

### Conclusion

Overall, the above demonstrates that silk-based NPs could serve as a biocompatible and inexpensive NO delivery platform. The amphiphilic properties of SF allow sustained release of NO by encapsulating the water-insoluble NO donor, SNAP, inside a hydrophobic core while having a more hydrophilic surface desired for cytocompatibility. The amide I band FTIR deconvolution confirmed the successful entrapment of SNAP molecules inside the core of SF NPs by hydrophobic interactions, which further resulted in increased crystallinity of SF NPs structure. SNAP-SF NPs were able to release NO in nanomolar range over a 24 hours period and exhibited strong antibacterial properties against MRSA and E. coli, which are responsible for several difficult-to-treat infections in humans. Besides, NPs did not show cytotoxic effects toward fibroblast cells at concentrations ≤2 mg/mL. SNAP-SF NPs either alone or when incorporated into other substrates can be potentially utilized for biomedical applications such as wound healing, where a combination of therapeutic and antibacterial properties of silk and NO are desired.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1. Yang, Y., et al., Nitric oxide based strategies for applications of biomedical devices. Biosurface and Biotribology, 2015. 1(3): p. 177-201.
2. Stewart, A. G., L. H. Phan, and G. Grigoriadis, Physiological and pathophysiological roles of nitric oxide. Microsurgery, 1994. 15(10): p. 693-702.
3. Liang, H., et al., Nitric oxide generating/releasing materials. Future science OA, 2015. 1(1).
4. Frost, M. C., M. M. Reynolds, and M. E. Meyerhoff, Polymers incorporating nitric oxide releasing/generating substances for improved biocompatibility of blood-contacting medical devices. Biomaterials, 2005. 26(14): p. 1685-1693.
5. Radi, R., Peroxynitrite, a stealthy biological oxidant. Journal of Biological Chemistry, 2013. 288(37): p. 26464-26472.
6. Vallance, P. and I. Charles, Nitric oxide as an antimicrobial agent: does NO always mean NO? Gut, 1998. 42(3): p. 313-314.
7. Witte, M. B. and A. Barbul, Role of nitric oxide in wound repair. The American Journal of Surgery, 2002. 183(4): p. 406-412.
8. Weller, R. B., Nitric oxide-containing nanoparticles as an antimicrobial agent and enhancer of wound healing. Journal of Investigative Dermatology, 2009. 129(10): p. 2335-2337.
9. Miller, M. and I. Megson, Recent developments in nitric oxide donor drugs. British journal of pharmacology, 2007. 151(3): p. 305-321.
10. Naghavi, N., et al., Nitric oxide donors for cardiovascular implant applications. Small, 2013. 9(1): p. 22-35.
11. Wang, P. G., T. B. Cai, and N. Taniguchi, Nitric oxide donors: for pharmaceutical and biological applications. 2005: John Wiley & Sons.
12. Jen, M. C., et al., Polymer-based nitric oxide therapies: Recent insights for biomedical applications. Advanced functional materials, 2012. 22(2): p. 239-260.
13. Brisbois, E. J., et al., Long-term nitric oxide release and elevated temperature stability with S-nitroso-N-acetylpenicillamine (SNAP)-doped Elast-eon E2As polymer. Biomaterials, 2013. 34(28): p. 6957-6966.
14. Seabra, A. B., et al., S-nitrosoglutathione incorporated in poly (ethylene glycol) matrix: potential use for topical nitric oxide delivery. Nitric Oxide, 2004. 11(3): p. 263-272.
15. Seabra, A., et al., Topically applied S-nitrosothiol-containing hydrogels as experimental and pharmacological nitric oxide donors in human skin. British Journal of Dermatology, 2004. 151(5): p. 977-983.
16. Georgii, J., et al., Topical S-nitrosoglutathione-releasing hydrogel improves healing of rat ischaemic wounds. Journal of tissue engineering and regenerative medicine, 2011. 5(8): p. 612-619.
17. Quinn, J. F., M. R. Whittaker, and T. P. Davis, Delivering nitric oxide with nanoparticles. Journal of controlled release, 2015. 205: p. 190-205.
18. Seabra, A. B. and N. Durán, Nitric oxide-releasing vehicles for biomedical applications. Journal of Materials Chemistry, 2010. 20(9): p. 1624-1637.
19. Lautner, G., M. E. Meyerhoff, and S. P. Schwendeman, Biodegradable poly (lactic-co-glycolic acid) microspheres loaded with S-nitroso-N-acetyl-D-penicillamine for controlled nitric oxide delivery. Journal of controlled release, 2016. 225: p. 133-139.
20. Nurhasni, H., et al., Nitric oxide-releasing poly (lactic-co-glycolic acid)-polyethylenimine nanoparticles for prolonged nitric oxide release, antibacterial efficacy, and in vivo wound healing activity. International journal of nanomedicine, 2015. 10: p. 3065.
21. D Marcato, P., et al., Development of a sustained-release system for nitric oxide delivery using alginate/chitosan nanoparticles. Current Nanoscience, 2013. 9(1): p. 1-7.
22. Zhang, Q. Y., et al., Gelatin-siloxane nanoparticles to deliver nitric oxide for vascular cell regulation: synthesis, cytocompatibility, and cellular responses. Journal of Biomedical Materials Research Part A, 2015. 103(3): p. 929-938.
23. Ghalei, S., H. Asadi, and B. Ghalei, Zein nanoparticle-embedded electrospun PVA nanofibers as wound dressing for topical delivery of anti-inflammatory diclofenac. Journal of Applied Polymer Science, 2018. 135(33): p. 46643.
24. Ghalei, S., et al., Enhanced cellular response elicited by addition of amniotic fluid to alginate hydrogel-electrospun silk fibroin fibers for potential wound dressing application. Colloids and Surfaces B: Biointerfaces, 2018. 172: p. 82-89.
25. Kundu, J., et al., Silk fibroin nanoparticles for cellular uptake and control release. International journal of pharmaceutics, 2010. 388(1-2): p. 242-250.
26. Seib, F. P., et al., pH-dependent anticancer drug release from silk nanoparticles. Advanced healthcare materials, 2013. 2(12): p. 1606-1611.
27. Rockwood, D. N., et al., Materials fabrication from Bombyx mori silk fibroin. Nature protocols, 2011. 6(10): p. 1612.
28. Chipinda, I. and R. H. Simoyi, Formation and stability of a nitric oxide donor: S-nitroso-N-acetylpenicillamine. The Journal of Physical Chemistry B, 2006. 110(10): p. 5052-5061.
29. Cao, Z., et al., The preparation of regenerated silk fibroin microspheres. Soft Matter, 2007. 3(7): p. 910-915.
30. Pignatelli, C., et al., Electrospun silk fibroin fibers for storage and controlled release of human platelet lysate. Acta biomaterialia, 2018. 73: p. 365-376.
31. Zhao, Z., Y. Li, and M.-B. Xie, Silk fibroin-based nanoparticles for drug delivery. International journal of molecular sciences, 2015. 16(3): p. 4880-4903.
32. Chen, M., Z. Shao, and X. Chen, Paclitaxel-loaded silk fibroin nanospheres. Journal of Biomedical Materials Research Part A, 2012. 100(1): p. 203-210.
33. Wang, J., et al., Natural Non-Mulberry Silk Nanoparticles for Potential-Controlled Drug Release. International journal of molecular sciences, 2016. 17(12): p. 2012.
34. Cebe, P., et al., Silk I and Silk ll studied by fast scanning calorimetry. Ada biomaterialia, 2017. 55: p. 323-332.
35. Singh, R. J., et al., Mechanism of nitric oxide release from S-nitrosothiols. Journal of Biological Chemistry, 1996. 271(31): p. 18596-18603.
36. Pant, J., et al., Tunable nitric oxide release from S-nitroso-N-acetylpenicillamine via catalytic copper nanoparticles for biomedical applications. ACS applied materials & interfaces, 2017. 9(18): p. 15254-15264.
37. Kevil, C. G. and R. P. Patel, S-nitrosothiol biology and therapeutic potential in metabolic disease. Current opinion in investigational drugs (London, England: 2000), 2010. 11(10): p. 1127.
38. Champeau, M., et al., Supramolecular poly (acrylic acid)/F127 hydrogel with hydration-controlled nitric oxide release for enhancing wound healing. Acta biomaterialia, 2018. 74: p. 312-325.
39. Schairer, D. O., et al., The potential of nitric oxide releasing therapies as antimicrobial agents. Virulence, 2012. 3(3): p. 271-279.
40. Pant, J., et al., A multi-defense strategy: Enhancing bactericidal activity of a medical grade polymer with a nitric oxide donor and surface-immobilized quaternary ammonium compound. Acta biomaterialia, 2017. 58: p. 421-431.
41. Backlund, C. J., B. V. Worley, and M. H. Schoenfisch, Anti-biofilm action of nitric oxide-releasing alkyl-modified poly (amidoamine) dendrimers against Streptococcus mutans. Acta biomaterialia, 2016. 29: p. 198-205.
42. Friedman, A., et al., Susceptibility of Gram-positive and-negative bacteria to novel nitric oxide-releasing nanoparticle technology. Virulence, 2011. 2(3): p. 217-221.

1. A biocompatible material comprising a nitric oxide donor embedded in silk fibroin nanoparticles.

2. The biocompatible material according to claim 1, wherein the nitric oxide donor is a S-nitrosothiol compound.

3. The biocompatible material according to claim 2, wherein the S-nitrosothiol compound is S-nitroso-N-acetyl-penicillamine, S-nitroso-N-acetyl cysteine, S-nitroso-N-acetyl cysteamine, S-nitrosoglutathione, methyl S-nitrosothioglycolate, or any combination thereof.

4. The biocompatible material according to claim 1, wherein the silk fibroin nanoparticles comprise a hydrophobic core, wherein a majority of the nitric oxide donor is present in the hydrophobic core of the silk fibroin nanoparticles.

5. The biocompatible material according to claim 1, wherein the silk fibroin nanoparticles have a diameter of about 300 nm to about 500 nm.

6. The biocompatible material according to claim 1, wherein the ratio of nitric oxide donor to silk fibroin nanoparticles is about 2:1 to about 1:4 by mass.

7. The biocompatible material according to claim 1, wherein a release half-life of the nitric oxide donor from the biocompatible material is about 2 hours to about 8 hours.

8. The biocompatible material according to claim 1, wherein the amount of nitric oxide donor release from the biocompatible material is about 0.010 nmol/mg to about 0.20 nmol/mg in about 24 hours.

9. The biocompatible material according to claim 1, wherein the nitric oxide donor is from about 5% by weight to about 15% by weight of the biocompatible material.

10. The biocompatible material according to claim 1, wherein the biocompatible material has a negative charge of about −25 mV to about −30 mV.

11. The biocompatible material according to claim 1, wherein the biocompatible material further comprises a drug.

12. A biocompatible material produced by the method comprising:
mixing a nitric oxide donor composition comprising a nitric oxide donor and a binary solvent with a silk fibroin to form a first composition comprising the biocompatible material; and
isolating the biocompatible material from the first composition.

13. The biocompatible material according to claim 12, further comprising centrifuging the first composition to obtain a sediment; and
freeze-drying the sediment to obtain a powder comprising silk fibroin nanoparticles embedded with the nitric oxide donor.

14. The biocompatible material according to claim 12, wherein the S-nitrosothiol compound is S-nitroso-N-acetyl-penicillamine, S-nitroso-N-acetyl cysteine, S-nitroso-N-acetyl cysteamine, S-nitrosoglutathione, methyl S-nitrosothioglycolate, or any combination thereof.

15. The biocompatible material according to claim 12, wherein the nitric oxide donor is S-nitroso-N-acetylpenicillamine.

16. The biocompatible material according to claim 12, wherein the binary solvent comprises an alcohol and water.

17. The biocompatible material according to claim 12, wherein the binary solvent comprises ethanol and water.

18. The biocompatible material according to claim 17, wherein a ratio of ethanol to water in the binary solvent is about 20:80 to about 40:60 by volume.

19. An article comprising the biocompatible material according to claim 1.

20. The article of claim 19, wherein the article is a wound dressing comprising a hydrogel.

21. The article of claim 19, wherein the article is a sponge.

22. A hydrogel comprising the biocompatible material according to claim 1.

23. The hydrogel of claim 22, wherein the hydrogel comprises alginate, gelatin, polyethylene glycol, polyvinyl alcohol, a poloxamer, or any combination thereof.
